(19) United States
(12) Patent Application Publication
(10) Pub. No.: US 2009/0259174 A1
(43) Pub. Date: Oct. 15, 2009

(54) Methods and devices for treating vulnerable atherosclerotic plaque

(75) Inventors: Michele Silver, Windsor, CA (US); Patrick E. Macaulay, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/103,423

(22) Filed: Apr. 15, 2008

(51) Int. Cl. A61M 1/00; A61M 25/14

(52) U.S. Cl. 604/28; 604/508; 604/35

(57) ABSTRACT

Methods and catheter devices/systems for removing some or all of the lipid core material from vulnerable plaque and/or for introducing one or more therapeutic substance into vulnerable plaque. The vulnerable plaque may be entered by a penetrator that is advanced into the vulnerable plaque from a catheter device that incorporates an on-board imaging, sensing or vulnerable plaque locating apparatus.

### FIELD OF THE INVENTION

The present invention relates generally to medical devices and methods, and more particularly to devices and methods for treatment of vulnerable atherosclerotic plaque.

### BACKGROUND

A vulnerable plaque is a type of atheromatous lesion in the wall of an artery. Vulnerable plaques are typically characterized by the presence of a soft lipid core covered by a relatively thin fiberous cap. While a vulnerable plaque is developing, its fiberous cap serves as a barrier between the soft lipid core material and blood that is flowing through the lumen of the artery. However, over time, the effects of continual hemodynamic pulsation of the artery and/or other mechanical stresses may cause the thin fiberous cap to tear, allowing the soft lipid core material to exude into the artery lumen and into contact with the flowing blood. This is known as plaque rupture. In some cases, plaque rupture may be facilitated by uncontrolled hypertension or periods of excessive exercise, excitation or exertion. Plaque rupture can result in various clinically significant sequale, as follows:
a) Plaque rupture can result in the formation of a localized blood clot on top of the site of the ruptured plaque resulting in acute blockage of the artery at the location of the ruptured plaque. Absent prompt intervention, this condition can result in ischemic damage or myocardial infarction downstream of the blockage.
b) Plaque rupture can result in spillage of some of the soft lipid core material and/or other debris into the blood stream such that it travels downstream and results in obstruction of smaller branch arteries or capillaries located downstream of the ruptured plaque. This may give rise to conditions known as “non-reflow” as well as ischemic tissue damage or myocardial infarction.
c) Plaque rupture may also allow a quantity of blood to collect within the vulnerable plaque lesion itself, thus causing the lesion to suddenly increase in size such that it protrudes into the artery lumen producing a partial or even total obstruction of the artery. Absent prompt intervention, this condition can also result in ischemic damage or myocardial infarction downstream of the blockage.

Vulnerable plaque can be difficult to detect. Often, the artery wall naturally thickens as the vulnerable plaque grows, preventing the plaque from abruptly protruding into the lumen of the artery. Rather, the artery lumen adjacent to a vulnerable plaque may remain fully patent and substantially unobstructed, although one side of its wall may be thickened due to the existence of the vulnerable plaque. Thus, the vulnerable plaque may be undetectable by traditional diagnostic tests that measure blood flow through the artery, such as cardiac stress tests and coronary angiography.

Various techniques have heretofore been proposed for treating or preventing the pathogenic rupture of vulnerable plaque. For example, U.S. Pat. No. 7,189,250 (DoBrava et al.) entitled Aspirating Balloon Catheter For Treating Vulnerable Plaque, which is expressly incorporated herein by reference, describes devices and methods for extracting core material contained in vulnerable plaque. In one embodiment, this method utilizes a catheter having an elongate shaft, a collection array of a plurality of collection lumens disposed about the distal portion of the elongate shaft, a means for radially extending and/or collapsing the collection array, and a suction means. The distal portion of the catheter is inserted into the blood vessel and the distal end of the collection array is positioned proximate the plaque deposit. The collection array is then extended to volitionally rupture the vulnerable plaque. The suction means is fluidly coupled to the proximal end of the collection array and is then used to suction away the core material that exudes from the ruptured plaque.

Also, U.S. Pat. No. 7,008,411 (Mandrusov, et al.) entitled Method and Apparatus For Treating Vulnerable Plaque, which is expressly incorporated herein by reference, describes a needle catheter adapted for insertion in and artery and useable to deliver biologically active agent to stabilize a vulnerable plaque. In at least one embodiment, the biologically active agent is one which strengthens the fibrous cap of the vulnerable plaque and the needle catheter is used in a way that avoids penetration into the soft lipid core of the vulnerable plaque. In another embodiment, therapeutic or biologically active agents to treat the vulnerable plaque may be delivered through the bloodstream or vessel wall. The therapeutic or biologically active agents disclosed in U.S. Pat. No. 7,008,411 (Mandrusov, et al.) include lipid lowering agents, antioxidants, extracellular matrix synthesis promoters, inhibitors of plaque inflammation and extracellular degradation, estradiol drug classes and their derivatives, proteins such as vascular endothelial growth factor (VEGF) in any of its multiple isoforms, fibroblast growth factors, monocyte chemoatractant protein 1 (MCP-1), transforming growth factor alpha (TGF-alpha), transforming growth factor beta (TGF-beta) in any of its multiple isoforms, DEL-1, insulin like growth factors (IGF), placental growth factor (PLGF), hepatocyte growth factor (HGF), prostaglandin E1 (PG-E1), prostaglandin E2 (PG-E2), tumor necrosis factor alpha (TBF-alpha), granulocyte stimulating growth factor (G-CSF), granulocyte macrophage colony-stimulating growth factor (GM-CSF), angiogenin, follistatin, and proliferin, genes encoding these proteins, cells transfected with these genes, pro-angiogenic peptides such as PR39 and PR11, and pro-angiogenic small molecules such as nicotine.

Additionally, U.S. Pat. No. 6,419,659 (Phelps et al.) entitled Lipid Pool Aspiration Arrangement For The Treatment Of Vulnerable Atherosclerosis Plaque, which is expressly incorporated herein by reference, describes a vulnerable plaque treatment catheter. In one embodiment, the catheter includes a flexible, steerable, lipid-enterable needle. The needle has at least one lumen that is in communication with a treatment source (e.g., a source of vacuum or a fluid infusion source) that is useable to render the lipid core into an innocuous entity. Where the treatment source comprises a vacuum source, the lipid core is aspirated through the lumen of the needle. Where the treatment source comprises a fluid infusion source, a fluid may be infused and combined with the lipid core to render it inert (e.g., to turn the lipid core into a non-thrombogenic semi-solid material). The catheter may include an optical fiber that is purportedly useable to view the vulnerable plaque and to monitor the procedure.

Also, United States Patent Application Publication No. 2005/0232965 (Folatico) entitled Local Administration Of A Combination Of Rapamycin And 17 Beta-Estradiol For The Treatment Of Vulnerable Plaque, which is expressly incorporated herein by reference, describes Medical devices, and in particular implantable medical devices, may be coated to minimize or substantially eliminate a biological organism's reaction to the introduction of the medical device to the organism. The medical devices may be coated with any number of biocompatible materials. Therapeutic drugs, agents or compounds may be mixed with the biocompatible materials and affixed to at least a portion of the medical device. These therapeutic drugs, agents or compounds may also further reduce a biological organism's reaction to the introduction of the medical device to the organism. In addition, these therapeutic drugs, agents and/or compounds may be utilized to promote healing, including the formation of blood clots. The drugs, agents, and/or compounds may also be utilized to treat specific diseases, including vulnerable plaque. Therapeutic agents may also be delivered to the region of a disease site. In regional delivery, liquid formulations may be desirable to increase the efficacy and deliverability of the particular drug. Also, the devices may be modified to promote endothelialization. Various materials and coating methodologies may be utilized to maintain the drugs, agents or compounds on the medical device until delivered and positioned. In addition, the devices utilized to deliver the implantable medical devices may be modified to reduce the potential for damaging the implantable medical device during deployment. Medical devices include stents, grafts, anastomotic devices, perivascular wraps, sutures and staples. In addition, various polymer combinations may be utilized to control the elution rates of the therapeutic drugs, agents and/or compounds from the implantable medical devices.

Additionally, United States Patent Application Publication No. 2006/0271154 (Woodall) entitled Methods And Systems For Treating Vulnerable Plaque describes methods and catheter-based delivery systems for treating vulnerable plaque atherosclerotic conditions using lightweight vulnerable plaque shields. Combination catheters are provided that include a selectively deployable occlusion balloon to occlude blood flow and a mechanism for selectively deploying a lightweight vulnerable plaque shield, which is either self-expanding or balloon-deployable, within a blood vessel.

There remains a need in the art for the development of new methods and devices for treating vulnerable plaque prior to its pathogenic rupture.

### SUMMARY OF THE INVENTION

The present invention provides methods and devices for removing some or all of the lipid core material from vulnerable plaque. Optionally, in some cases, the present invention further provides methods and devices for depositing therapeutic substance(s) into the interior of the vulnerable plaque after some or all of its lipid core has been removed.

In accordance with one aspect of the present invention, there is provided a method for treating vulnerable plaque comprising the steps of (A) providing a catheter device that comprises a catheter body, a vulnerable plaque locating element and a penetrator that has a lumen, said penetrator being advanceable laterally from the catheter body; (B) positioning the catheter body in the artery; (C) using the vulnerable plaque locating element to locate the vulnerable plaque; (D) positioning and rotationally orienting the catheter such that subsequent advancement of the penetrator will cause the penetrator to enter the vulnerable plaque; (E) advancing the penetrator into the vulnerable plaque; and (F) performing at least one procedure selected from the group consisting of: (i) aspirating matter out of the vulnerable plaque and (ii) introducing a therapeutic substance into the vulnerable plaque. In some embodiments, Step F may comprise applying suction and/or injecting a therapeutic substance through the lumen of the penetrator so as to aspirate matter out of the vulnerable plaque and into the lumen of the penetrator. In other embodiments, a single lumen catheter such as a microcatheter may be advanced through the lumen of the penetrator and suction may then be applied and/or a therapeutic substance may be delivered, through the lumen of that catheter so as to aspirate matter out of the vulnerable plaque and into the lumen of that catheter. In some cases, where matter is aspirated from the vulnerable plaque lesion and therapeutic substance is introduced into the vulnerable plaque lesion, one of those procedures may be performed directly through the lumen of the penetrator and the other may be performed through the lumen of a catheter (e.g., a microcatheter) that is inserted through the lumen of the penetrator. In this regard, a catheter may be initially advanced through the lumen of the penetrator and a quantity of the soft lipid core material may be aspirated into the lumen of that catheter. Thereafter, that catheter may be removed and a therapeutic substance may then be introduced directly through the lumen of the penetrator and into the vulnerable plaque.

Further in accordance with the present invention, in cases where the above described method includes both the aspiration of matter from the vulnerable plaque and the introduction of a therapeutic substance into the vulnerable plaque, the therapeutic substance may be deposited into a void or space that has been created by the prior aspiration of matter (e.g., lipid core material) from the vulnerable plaque. In some embodiments, the therapeutic substance may be delivered directly through the lumen of the penetrator. In other embodiments, a catheter such as a microcatheter may be advanced through the lumen of the penetrator and the therapeutic substance may then be delivered through that catheter and into the vulnerable plaque.

Further aspects, elements, embodiments, objects and advantages of the present invention will be appreciated by those of skill in the relevant art upon reading the detailed description and examples set forth herebelow.

### BRIEF DESCRIPTION OF THE DRAWINGS

FIG. 1 is a perspective view of one embodiment of a vulnerable plaque treating catheter system of the present invention.

FIG. 1A is an enlarged view of area 1A of FIG. 1.

FIG. 1B is a cross sectional view through line 1B-1B of FIG. 1.

FIG. 2 is a cut-away diagram of a vulnerable plaque lesion in the wall of an artery.

FIGS. 3A-3H show a series of steps in one embodiment of a method for treating vulnerable plaque in accordance with the present invention.

FIG. 4A shows an intravascular ultrasound (IVUS) image received from an IVUS transducer located on the tissue penetrating catheter wherein a penetrator path indicator indicates that the catheter is in an incorrect rotation orientation and, as a result, advancement of the penetrator while the catheter is in its present orientation would cause the penetrator to enter the wall of the artery at a location other than the vulnerable plaque lesion.

FIG. 4B shows an IVUS image received from the tissue penetrating catheter of FIG. 4A after the catheter body had been rotated to a correct rotational orientation wherein the penetrator path indicator indicates that advancement of the penetrator while the catheter is in its present orientation will cause the penetrator to enter the vulnerable plaque lesion as intended.

### DETAILED DESCRIPTION AND EXAMPLES

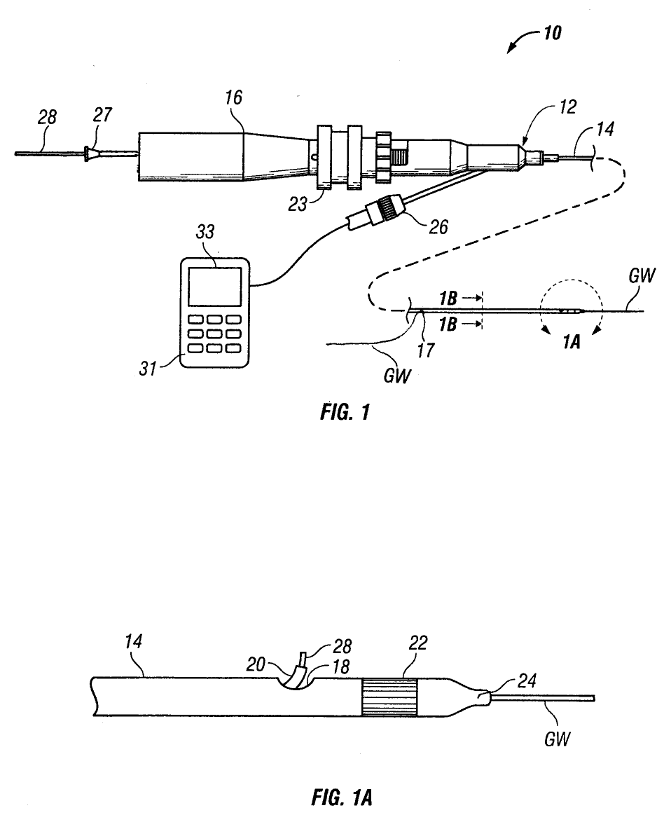

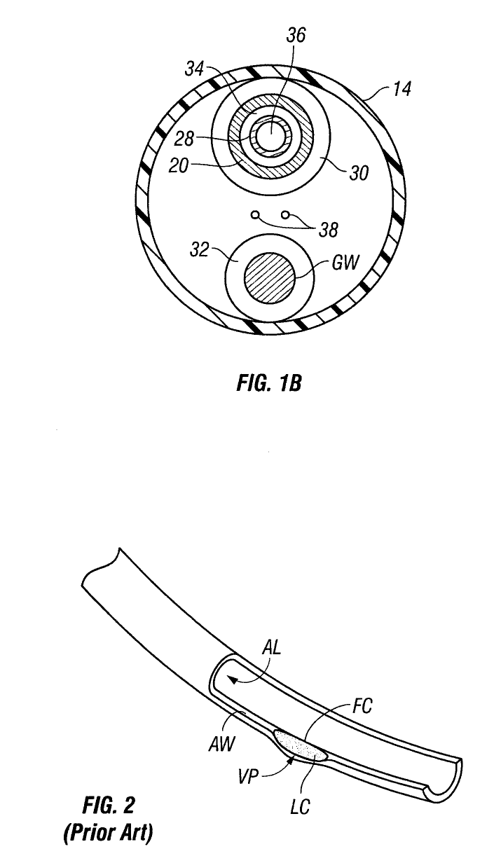

FIGS. 1, 1A and 1B show a catheter system 10 that is useable to treat vulnerable plaque. This catheter system 10 comprises a tissue penetrating catheter 12 that has an elongate flexible catheter body 14 and a handpiece 16 on the proximal end of the catheter body 14. A tissue penetrating catheter of this type is commercially available as the Pioneer™ Catheter from Medtronic Vascular, Inc., Santa Rosa, Calif. Further details and examples of the construction and operation of the tissue penetrating catheter 12 and alternative designs and embodiments thereof are also described in various publications including U.S. Pat. No. 6,746,464 (Makower), U.S. Pat. No. 6,726,677 (Flaherty et al.), U.S. Pat. No. 6,709,444 (Makower), U.S. Pat. No. 6,669,709 (Cohen et al.), U.S. Pat. No. 6,685,716 (Flaherty et al.), U.S. Pat. No. 6,685,648 (Flaherty et al.), U.S. Pat. No. 6,660,024 (Flaherty et al.), U.S. Pat. No. 6,579,311 (Makower), U.S. Pat. No. 6,561,998 (Roth et al.), U.S. Pat. No. 6,379,319 (Garibotto et al.), U.S. Pat. No. 6,375,615 (Flaherty et al.), U.S. Pat. No. 6,302,875 (Makower et al.), U.S. Pat. No. 6,283,983 (Makower et al.), U.S. Pat. No. 6,190,353 (Makower et al.), U.S. Pat. No. 6,159,225 (Makower), U.S. Pat. No. 6,068,638 (Makower) and U.S. Pat. No. 5,830,222 (Makower), the entire disclosures of such patents being expressly incorporated herein by reference.

As seen in the cross sectional showing of FIG. 1B, the tissue penetrating catheter 12 has a penetrator lumen 30 and a guidewire lumen 32 extending through the catheter body 14. The penetrator lumen 30 terminates distally in a penetrator exit port 18 located on the side of the catheter body 14. A penetrator 20 is initially positioned in a retracted position within the penetrator lumen 30 and is thereafter advanceable out of the penetrator outlet port 18 as indicated in FIG. 1A. A penetrator control 23 on the handpiece 16 may be used by the operator to effect the advancement and retraction of the penetrator 20, when desired. This penetrator control 23 may operate to advance the penetrator in discernible measured increments (e.g., 1 millimeter per click) and/or may include a scale, gage or measurement indicia that indicates to the operator the distance to which the penetrator 20 has advanced from the catheter body 14. In this regard, in treating a typical vulnerable plaque lesion in a coronary artery of a normal adult human, it will typically be desirable to advance about 1 to 5 millimeters of the penetrator 20 out of the catheter body 14 to ensure that the distal end of the penetrator 20 has penetrated through the fibrous cap of the vulnerable plaque and into the soft lipid core of the lesion but has not traveled all the way through the soft lipid core.

In the particular embodiment of the catheter system 10 shown in the drawings of this patent application, the penetrator 20 comprises hollow, curved needle having a penetrator lumen 34 extending therethrough. A proximal port 27 on handpiece 16 leads into the penetrator lumen 34 such that matter may be aspirated or infused through the penetrator lumen 34 and, in some cases, a separate catheter such as a small microcatheter may be advanced through the penetrator lumen 34.

When intended for use in performing procedures in the coronary arteries of normal, average-sized adult humans via a radial artery or femoral artery approach, the catheter body 14 may be in the range of 75-130 cm in length and a distal portion of the catheter body may optionally incorporate a reinforcement member such as a wire braid. Such wire braid may terminate approximately 3 cm from the distal end of the catheter body 14. The procedure is also useable at other locations (e.g., arteries other than coronary arteries) in the body and other length catheters from 10-150 cm could be used depending on the site at which the catheter enters the body and the location of the vulnerable plaque to be treated. Also, optionally, one or more radiopaque marker(s) may be positioned at desired location(s) on the catheter body 14, such as at the distal end of the catheter and/or adjacent to the penetrator outlet port 18.

In a rapid-exchange embodiment of the tissue penetrating catheter 12 as shown in FIG. 1, the guidewire lumen 32 may extend through a distal portion of the catheter body 14 from a proximal guidewire port 17 located on the sidewall of the catheter body through an opening in the distal tip 24, as shown. Alternatively, an over-the-wire embodiment of the tissue penetrating catheter 12 may also be provided, wherein the guidewire lumen 32 extends through the entire catheter from a guidewire port on the handpiece 16 to the opening in the distal tip 24.

Also, in this example, the tissue penetrating catheter 12 the distal tip section 24 is optionally tapered and formed of material that is softer and more flexible than the adjacent distal portion of the catheter body 14.

### On Board Imaging/Sensing/Guidance

In the particular embodiment of the penetrating catheter 12 shown, an on-board imaging transducer 22 is positioned on the catheter body 14. In this embodiment, the imaging transducer 22 comprises a phased array IVUS transducer that is operative to image 360° around the catheter body 14. This imaging transducer 22 comprises an annular array of individual crystals or elements coupled to a multiplex circuit which is within the catheter body 14. The multiplex circuit is in turn coupled to leads 38 which extend through the catheter body 14 and into side arm connector 26 on handpiece 16. This side arm connector 26 is connectable to an imaging console 31 which houses the apparatus required for IVUS imaging as well known in the art. When activated, the imaging transducer 22 emits ultrasound signals and receives back echoes or reflections which are representative of the nature of the surrounding environment. The imaging transducer 22 provides an imaging signal from which an image of the surrounding structures can be created by signal processing apparatus located in an imaging console 31 and displayed on screen 33. Additionally, this imaging transducer 22 may provide on the screen 33 indicia of high stress areas of the vascular wall and low stress areas of the vascular wall. For example, the signal processing circuitry of the imaging console 31 may be capable of color coding low stress and high stress areas on the displayed image. Low stress areas will typically be associated with normal arterial wall while high stress areas may be associated with the thin fibrous cap covering the lipid core of a vulnerable plaque lesion. A suitable IVUS phased array transducer 22 as well as the accompanying image processing apparatus/circuitry and imaging console 31 may be obtained commercially from Volcano Therapeutics, Inc., Laguna Hills, Calif.

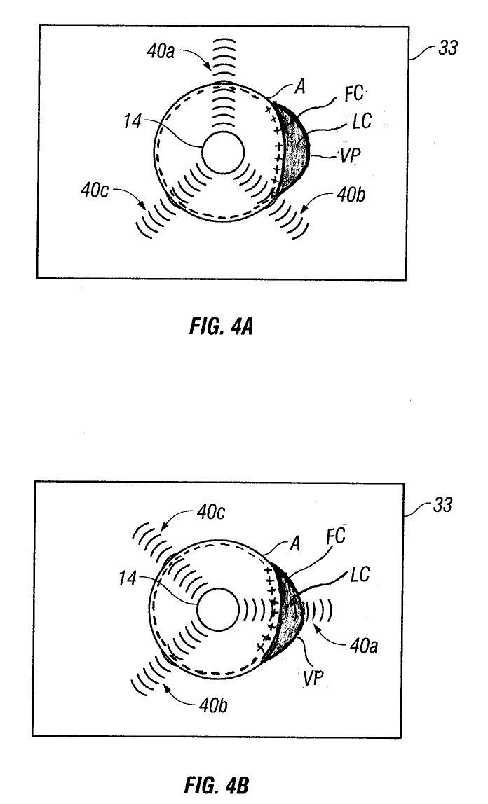

As described in detail in the above-incorporated U.S. Pat. No. 6,746,464 (Makower), U.S. Pat. No. 6,726,677 (Flaherty et al.), U.S. Pat. No. 6,709,444 (Makower), U.S. Pat. No. 6,669,709 (Cohen et al.), U.S. Pat. No. 6,685,716 (Flaherty et al.), U.S. Pat. No. 6,685,648 (Flaherty et al.), U.S. Pat. No. 6,660,024 (Flaherty et al.), U.S. Pat. No. 6,579,311 (Makower), U.S. Pat. No. 6,561,998 (Roth et al.), U.S. Pat. No. 6,379,319 (Garibotto et al.), U.S. Pat. No. 6,375,615 (Flaherty et al.), U.S. Pat. No. 6,302,875 (Makower et al.), U.S. Pat. No. 6,283,983 (Makower et al.), U.S. Pat. No. 6,190,353 (Makower et al.), U.S. Pat. No. 6,159,225 (Makower), U.S. Pat. No. 6,068,638 (Makower) and U.S. Pat. No. 5,830,222 (Makower), an imageable marker structure may be mounted on or in the catheter body 14 in a known circumferential orientation relative to the penetrator exit port 18 or to the trajectory on which the penetrator 20 advances out of exit port 18. With specific reference to FIGS. 4A and 4B (described in more detail herebelow), this imageable marker structure may include three struts or members that create three visible images 40a, 40b and 40c on the image and/or indicia of a high stress region on the artery wall, received from the imaging transducer 22. One of these visible images 40a is distinguishable from the other two images 40b and 40c and serves as an indicator of the expected path on which the penetrator 20 will subsequently advance from the catheter body 14 relative to the location of the vulnerable plaque lesion VP (as visible on the IVUS image of the VP lesion itself and/or as indicated by IVUS detection of an area of high stress on the artery wall). As explained in the example below, this penetrator path predicting image 40a may be used by the operator to adjust not only the longitudinal position of the catheter body 14 relative to the vulnerable plaque VP but also the rotational orientation of the catheter body 14 within the artery A to ensure that when the penetrator 20 is subsequently advanced it will penetrate into the vulnerable plaque lesion as desired and not into some other location on the artery wall.

It will be appreciated that, as an alternative to the use of a physical marker structure, the imaging transducer 22 could be mounted in a fixed position and a selected one (or selected ones) of the individual imaging elements (e.g., crystals) of the phased array may be selected as being in longitudinal alignment with the penetrator exit port 18 or otherwise located so as to be indicative of the trajectory or path on which the penetrator 20 will subsequently advance from the catheter body 14. The selected imaging element(s) will thus serve as penetrator-path-indicating imaging element(s) and will be electronically identified so as to form a visual penetrator path indicator (e.g., a line or pointer) on the screen 33 of the imaging console 31. Additionally, as discussed

In some embodiments, as an alternative to an IVUS imaging transducer 22 or other imaging device or sensor may be used to image or sense or otherwise determine the location of the vulnerable plaque and to effectively aim the penetrator 20 such that when the penetrator 20 is advanced, it will penetrate through the fibrous cap of the vulnerable plaque. For example, as an alternative to, or in addition to, and IVUS imaging transducer 22, the catheter may be equipped with an intravascular magnetic resonance apparatus useable to image and/or locate vulnerable plaque as described in Atalar, E. et al.: High Resolution Intravascular MRI and MRS by Using a Catheter Receiver Coil; Magnetic Resonance in Medicine, 36:596-605 (1996); Rivas, P. et al.: In Vivo Real-Time Intravascular MRI; Journal of Cardiovascular Magnetic Resonance, 4(2);223-232 (2002) and Susil, R., et al.: Intravascular Extended Sensitivity (IVES) MRI Antennas: Magnetic Resonance in Medicine, 50:383-390 (2003). Also, examples of the types of sensors or vulnerable plaque locating devices that may be used as an alternative to, or in addition to IVUS transducer 22, include temperature sensors, thermography systems, illuminators/phase discrimination systems, optical diffraction systems, palpographic systems, angioscopes, near infrared spectroscopy systems, optical coherence tomography systems, x-ray angiography systems, multidetector row computed angiography systems, calcium measuring or detecting systems and other technologies that sense localized increases in temperature, tissue density or other variables that distinguish vulnerable plaque from adjacent tissue. These alternative types of sensors or vulnerable plaque locating devices include but are not limited to those described in Hamdan, A., et al.; Imaging of Vulnerable Coronary Artery Plaques; Catheterization and Coronary Artery Interventions 70:65-74 (2007) as well as U.S. Pat. No. 7,313,432 (Tearney) entitled Phase Discrimination For Detection Of Vulnerable-Plaque; U.S. Pat. No. 7,297,154 (Tu et al.) entitled Optical apparatus for detecting and treating vulnerable plaque; U.S. Pat. No. 7,288,244 (Van Langenhove, et al.) entitled Determining Vulnerable Plaque In Blood Vessels; U.S. Pat. No. 7,195,599 (Carney et al.) entitled Instrumented Catheter With Distance Compensation To Sense Vulnerable Plaque; U.S. Pat. No. 7,077,812 (Naghavi) entitled Apparatus And Method For Palpographic Characterization Of Vulnerable Plaque and other biological tissue; U.S. Pat. No. 6,579,243 (Kokate et al.) entitled Catheter With Thermal Sensor For Detection Of Vulnerable Plaque and United States Patent Application Publications No. 2007/0166231 (Agah) entitled Methods And Probes For Identifying Vulnerable Plaque; 2007/0076212 (Zuluaga) entitled Detecting Vulnerable Plaque and 2005/0075574 (Furnish et al.) entitled Devices For Vulnerable Plaque Detection, the entire disclosure of each such patent and patent application being expressly incorporated herein by reference.

Additionally, in some embodiments, the tissue penetrating catheter 12 may incorporate or may be used in conjunction with a distal protection device such as a balloon, umbrella like device, mesh screen or other apparatus that captures or prevents particles of solid matter that may be released during the procedure from migrating downstream through the lumen of the artery being treated.

To facilitate description and understanding of the methods by which the above-described catheter system 10 may be used to treat vulnerable plaque in accordance with the present invention, FIG. 2 shows a cut-away view of an artery A having an artery wall AW, and artery lumen AL and a vulnerable plaque lesion VP in the artery wall AW. As shown, the vulnerable plaque VP comprises a soft lipid core LC covered by a fibrous cap FC.

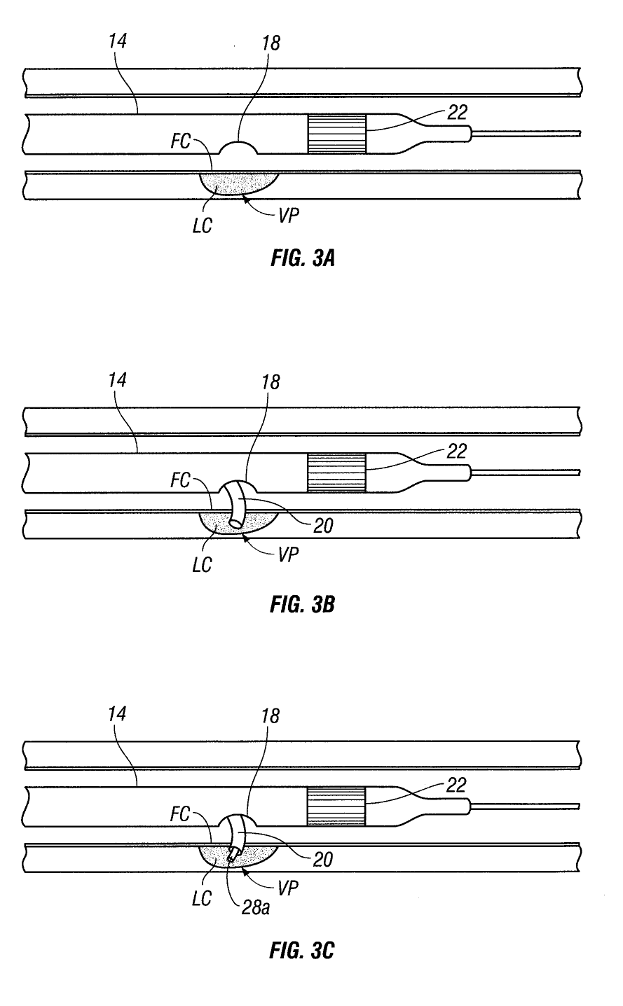

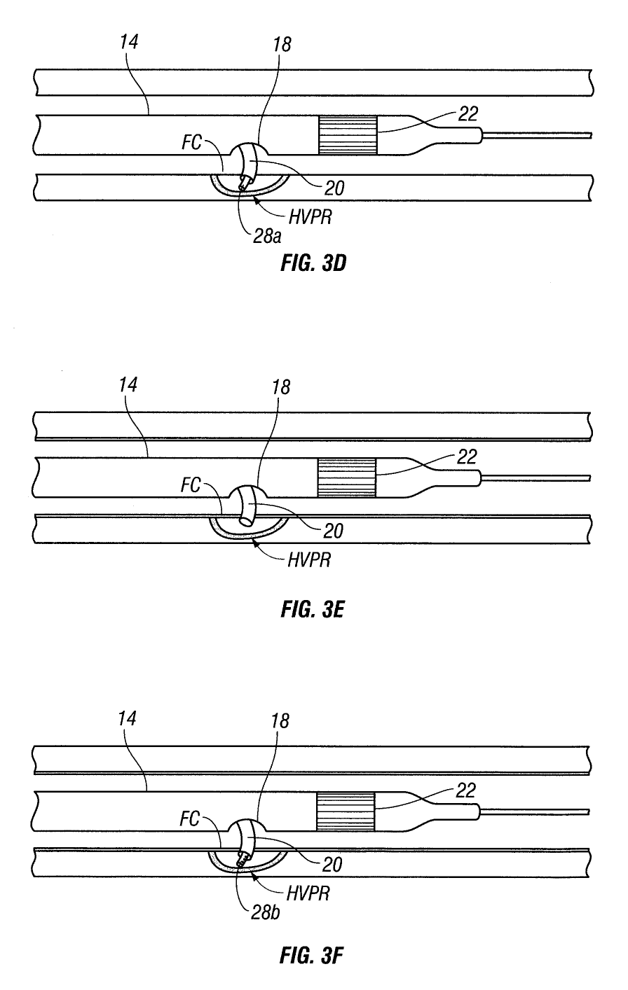

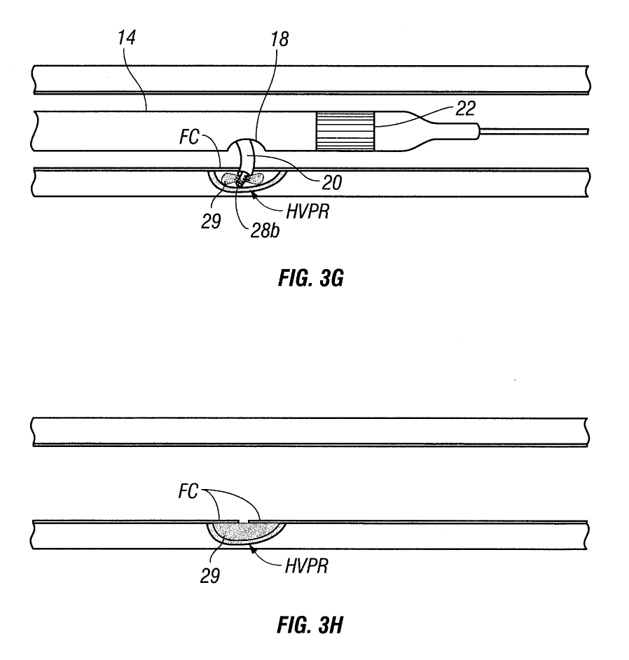

FIGS. 3A-3H show one example of a method for treating vulnerable plaque in accordance with this invention. As seen in FIG. 3A, the tissue penetrating catheter body 14 is advanced into the lumen of an artery. The imaging transducer 22 and penetrator path indicating marker structure (not shown) are used to obtain an IVUS image of the artery wall A and the vulnerable plaque, and the longitudinal position and rotational orientation of the catheter body 14 are adjusted as necessary to juxtaposition the penetrator outlet port 18 to the vulnerable plaque VP so that subsequent advancement of the penetrator 20 will cause the penetrator 20 to enter the vulnerable plaque VP. In this regard, FIG. 4A shows an example of an IVUS image that would appear on the imaging console screen 33 when the catheter body 14 is not in the proper rotational orientation. On this IVUS image, the operator can see an image of the vulnerable plaque VP lesion, including its lipid core LC and fibrous cap FC. Optionally, this IVUS image may also include color coding or other indicia of artery wall stress so as to show an area of high stress (indicated by plus signs ++++ on FIGS. 4A and 4B) where the thin fibrous cap FC is located and an area of low stress (indicated by minus signs −−−− on FIGS. 4A and 4B). As shown in FIG. 4A, the penetrator path indicator 40a is directed from the image of the catheter body 14 into a location on the artery wall that is away from the vulnerable plaque VP lesion and away for the area of high stress (+++), thus indicating that the catheter 14 is not in the correct rotational orientation. While observing the screen 33, the operator then rotates the catheter body 14 within the artery A until the penetrator path indicator 40a extends from the catheter body 14 into the vulnerable plaque VP (e.g., into the center of the visible image of the vulnerable plaque lesion VP and/or into the center of the area of high wall stress (+++), as shown in FIG. 4B. The catheter body is now in the proper rotational orientation and the operator may continue with the treatment procedure.

As seen in FIG. 3B, the penetrator 20 is then advanced out of penetrator outlet port 18, through the fibrous cap FC and into the soft lipid core LC of the vulnerable plaque VP. At this stage, suction could be applied through the penetrator lumen to aspirate a quantity of soft lipid core LC from the vulnerable plaque VP and/or a therapeutic or diagnostic substance could be injected directly through the penetrator lumen. Instead however, in the particular example shown in FIG. 3C, a first microcatheter 28a is inserted through port 27 and is advanced through the penetrator lumen 34 and out of its distal end of the penetrator 20 so that the distal end of the microcatheter 28a is within the soft lipid core LC of the vulnerable plaque VP. Optionally, in some patients, a lipid dissolving or lypolytic substance may be injected into the lipid core LC through the first microcatheter 28a to dissolve or modify some or all of the lipid material in a manner that lowers its viscosity in preparation for aspiration. Types of material that could be injected could include Phosphatidylcoline Deoxycholate (Lipodissolve), emulsifiers (Polysorbate 20), Lecithin, natural occurring enzymes or lypase such as bile or pancreatic lipase, or components of snake or spider venom (ex: hyaluronidase, amino acid oxidases & proteases, sphingomyelinase D). After any optional lipid dissolution of lipolysis has been been carried out, suction is applied to the first microcatheter 28a to aspirate a quantity of the soft lipid core LC out of the vulnerable plaque and into the lumen of the first microcatheter 28a. This converts the vulnerable plaque VP into a hollow vulnerable plaque remnant HVPR. In some embodiments, the procedure may end at this point, the first microcatheter 28a may be withdrawn into the penetrator 20, the penetrator 20 may be retracted into the catheter body 14 and the penetrating catheter 12 may be removed from the subject's body. Because a substantial quantity of the lipid core LC has been removed, the hollow ventricular plaque remnant HVPR no longer presents a risk of plaque rupture. However, in other embodiments, the procedure may proceed further with the delivery of a therapeutic substance into the hollow vulnerable plaque remnant HVPR, as shown in FIGS. 3E through 3H.

As shown in FIG. 3E, in order to proceed with the optional therapeutic substance delivery, the first microcatheter 28a is withdrawn and removed, leaving the penetrator 20 deployed within the hollow vulnerable plaque remnant HVPR. At this point, the operator could elect to infuse the desired therapeutic substance directly through the penetrator lumen 34 and into the interior of the hollow vulnerable plaque remnant HVPR. However, in this example, the operator has opted instead to utilize a second microcatheter 28b. Accordingly, as shown in FIG. 3F, a second microcatheter 28b is inserted through proximal port 27 and advanced through the penetrator lumen 34 until a small portion of the distal end of the second microcatheter 28b protrudes out of the penetrator 20 within the interior of the hollow vulnerable plaque remnant HVPR. Thereafter, as seen in FIG. 3G, a quantity of a therapeutic substance 29 is infused through the second microcatheter 28b and into the hollow vulnerable plaque remnant HVPR. Examples of the types of therapeutic substance 29 that may be delivered include but are not limited to fillers, anticoagulants, heparin; gene therapy preparations; vulnerable plaque stabilizing agents; agents which strengthen a fibrous cap of the vulnerable plaque; steroids, antiproliferative agents, potassium channel inhibitors, rapamycin, sirolomus, paclitaxil, rapamycin in combination with 17 beta-estradiol; lipid lowering agents; antioxidants; extracellular matrix synthesis promoters; inhibitors of plaque inflammation and extracellular degradation; estradiol drug classes and their derivatives; proteins; vascular endothelial growth factor (VEGF) in any of its multiple isoforms; fibroblast growth factors; monocyte chemoatractant protein 1 (MCP-1); transforming growth factor alpha (TGF-alpha); transforming growth factor beta (TGF-beta) in any of its multiple isoforms; DEL-1, insulin like growth factors (IGF); placental growth factor (PLGF); hepatocyte growth factor (HGF); prostaglandin E1 (PG-E1); prostaglandin E2 (PG-E2); tumor necrosis factor alpha (TBF-alpha); granulocyte stimulating growth factor (G-CSF); granulocyte macrophage colony-stimulating growth factor (GM-CSF); angiogenin; follistatin; proliferin; genes encoding angiogenin, follistatin or proliferin; cells transfected with genes encoding angiogenin, follistatin or proliferin; pro-angiogenic peptides; PR39; PR11; pro-angiogenic small molecules and nicotine.

Finally, in this example, after the therapeutic substance has been delivered, the second microcatheter 28b is withdrawn into the penetrator 20, the penetrator 20 is retracted into the catheter body 14 and the penetrating catheter 12 is removed from the subject's body.

As those of skill in the art will appreciate, after completion of the removal of all or part of the lipid core LC and optionally the introduction of a therapeutic substance 29 as described in the above-set-forth example, a stent, stent-graft or covered stent may be implanted in the artery A adjacent to the hollow vulnerable plaque remnant HVPR (with or without a quantity of therapeutic substance 29 therewithin) to provide further stability and/or to minimize the likelihood that matter will escape into the bloodstream through the opening that has been created in the fibrous cap FC.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A method for treating vulnerable plaque in an artery of a human or animal subject, said method comprising the steps of:
(A) providing a catheter device that comprises a catheter body, a vulnerable plaque locating element and a penetrator that has a lumen, said penetrator being advanceable laterally from the catheter body;
(B) positioning the catheter body in the artery;
(C) using the vulnerable plaque locating element to locate the vulnerable plaque;
(D) positioning and rotationally orienting the catheter such that subsequent advancement of the penetrator will cause the penetrator to enter the vulnerable plaque;
(E) advancing the penetrator into the vulnerable plaque; and
(F) performing at least one procedure selected from the group consisting of: (i) aspirating matter out of the vulnerable plaque and (ii) introducing a therapeutic substance into the vulnerable plaque.

2. A method according to claim 1 wherein Step F comprises applying suction through the lumen of the penetrator so as to aspirate matter out of the vulnerable plaque and into the lumen of the penetrator.

3. A method according to claim 1 wherein Step F comprises introducing a therapeutic substance through the lumen of the penetrator.

4. A method according to claim 1 wherein Step F comprises:
i. advancing a catheter having a lumen through the lumen of the penetrator;
ii. applying suction through the lumen of the catheter that has been advanced through the lumen of the penetrator so as to aspirate matter out of the vulnerable plaque and into the lumen of the catheter.

5. A method according to claim 1 wherein Step F comprises:
i. advancing a catheter having a lumen through the lumen of the penetrator;
ii. introducing a therapeutic substance through the lumen of the catheter that has been advanced through the lumen of the penetrator.

6. A method according to claim 1 wherein Step F comprises aspirating matter out of the vulnerable plaque and, thereafter, introducing a therapeutic substance into the vulnerable plaque.

7. A method according to claim 6 wherein a catheter is advanced through the lumen of the penetrator and matter is aspirated from the vulnerable plaque into the lumen of that catheter and, thereafter, that catheter is removed and thereafter introducing the therapeutic substance through the lumen of the penetrator and into the vulnerable plaque.

8. A method according to claim 7 wherein the therapeutic substance is introduced into a void or space that was created within the prior vulnerable plaque by the aspiration of matter therefrom.

9. A method according to claim 1 wherein Step F comprises:
i. advancing a first catheter having a lumen through the lumen of the penetrator;
ii. applying suction through the lumen of the first catheter so as to aspirate matter out of the vulnerable plaque and into the lumen of the first catheter.

10. A method according to claim 1 wherein step F comprises aspirating matter out of the vulnerable plaque and the method further comprises the step of injecting a lipid dissolving or lipolytic substance to dissolve or otherwise lower the viscosity of matter contained within the vulnerable plaque prior to applying suction.

11. A method according to claim 10 wherein the lipid dissolving or lipolytic substance comprises at least one substance selected from the group consisting of: Phosphatidylcoline Deoxycholate, emulsifiers, Polysorbate 20, Lecithin, natural occurring enzymes, lypases, bile lipase, pancreatic lipase, components of snake or spider venom, hyaluronidase, amino acid oxidases, amino acid proteases and sphingomyelinase D.

12. A method according to claim 9 wherein Step G comprises:
i. removing the first catheter from the penetrator lumen;
ii. advancing a second catheter having a lumen through the lumen of the penetrator; and
iii. introducing the therapeutic substance through the lumen of the second catheter and into the vulnerable plaque.

13. A method according to claim 1 wherein a therapeutic substance is introduced into the vulnerable plaque in Step F and wherein the therapeutic substance is selected from the group consisting of: fillers, anticoagulants, heparin; gene therapy preparations; vulnerable plaque stabilizing agents; agents which strengthen a fibrous cap of the vulnerable plaque; steroids, antiproliferative agents, potassium channel inhibitors, rapamycin, sirolomus, paclitaxil, rapamycin in combination with 17 beta-estradiol; lipid lowering agents; antioxidants; extracellular matrix synthesis promoters; inhibitors of plaque inflammation and extracellular degradation; estradiol drug classes and their derivatives; proteins; vascular endothelial growth factor (VEGF) in any of its multiple isoforms; fibroblast growth factors; monocyte chemoatractant protein 1 (MCP-1); transforming growth factor alpha (TGF-alpha); transforming growth factor beta (TGF-beta) in any of its multiple isoforms; DEL-1, insulin like growth factors (IGF); placental growth factor (PLGF); hepatocyte growth factor (HGF); prostaglandin E1 (PG-E1); prostaglandin E2 (PG-E2); tumor necrosis factor alpha (TBF-alpha); granulocyte stimulating growth factor (G-CSF); granulocyte macrophage colony-stimulating growth factor (GM-CSF); angiogenin; follistatin; proliferin; genes encoding angiogenin, follistatin or proliferin; cells transfected with genes encoding angiogenin, follistatin or proliferin; pro-angiogenic peptides; PR39; PR11; pro-angiogenic small molecules and nicotine.

14. A system for treating a vulnerable plaque that has a lipid core and a fibrous cap located in an artery of a human or animal subject, said system comprising:
a tissue penetrating catheter comprising a) an elongate catheter body that is insertable into the artery, b) a penetrator having a lumen, said penetrator being advanceable laterally from the catheter body and c) an orientation apparatus;
the orientation apparatus being operative to provide a prediction of the penetrator path relative to the location of the vulnerable plaque to facilitate adjustment of the rotational orientation of the catheter body within the artery prior to advancement of the penetrator to substantially ensure that, when the penetrator is subsequently advanced, it will penetrate into the vulnerable plaque rather than some other location on the artery;
the distance of advancement of the penetrator being controllable so that, when advanced, the penetrator will penetrate through the fibrous cap and into, but not all the way through, the lipid core of the vulnerable plaque;
at least one procedure being thereafter performable via the lumen of the penetrator, said at least one procedure being selected from the group consisting of: (i) aspirating a quantity of matter from the vulnerable plaque and (ii) introducing a therapeutic substance into the vulnerable plaque.

15. A system according to claim 14 further comprising:
a first catheter having a lumen, said first catheter being advanceable through the lumen of the penetrator; and
at least one of (i) a source of negative pressure connectable to the lumen of the first catheter to aspirate a quantity of lipid core material into the lumen of the first catheter and (ii) a source of therapeutic substance connectable to the lumen of the first catheter for delivery of the therapeutic substance through the lumen of the first catheter.

16. A system according to claim 15 wherein a source of negative pressure is connected to the lumen of the first catheter to aspirate a quantity of lipid core material into the lumen of the first catheter and wherein the system further comprises:
a source of a lipid dissolving or lypolitic agent that is connected to the lumen of the first catheter and from which a quantity of the lipid dissolving or lypolitic agent is injected through the lumen of the first catheter to reduce the viscosity of matter contained within the vulnerable plaque prior to connection of the source of negative pressure to the lumen of the first catheter.

17. A system according to claim 16 wherein the source of a lipid dissolving or lypolitic agent comprises a source of at least one agent selected from the group consisting of: Phosphatidylcoline Deoxycholate, emulsifiers, Polysorbate 20, Lecithin, natural occurring enzymes, lypases, bile lipase, pancreatic lipase, components of snake or spider venom, hyaluronidase, amino acid oxidases, amino acid proteases and sphingomyelinase D.

18. A system according to claim 15 or 16 wherein a source of negative pressure is connected to the lumen of the first catheter to aspirate a quantity of lipid core material into the lumen of the first catheter and the first catheter is subsequently removable from the penetrator lumen and wherein the system further comprises:
a second catheter having a lumen, said second catheter being advanceable through the lumen of the penetrator after the first catheter has been removed therefrom; and
a source of therapeutic substance that is connectable to the lumen of the second catheter for delivery of the therapeutic substance through the lumen of the second catheter.

19. A system according to claim 18 wherein the source of therapeutic substance comprises a source of at least one substance selected from the group consisting of: fillers, anticoagulants, heparin; gene therapy preparations; vulnerable plaque stabilizing agents; agents which strengthen a fibrous cap of the vulnerable plaque; steroids, antiproliferative agents, potassium channel inhibitors, rapamycin, sirolomus, paclitaxil, rapamycin in combination with 17 beta-estradiol; lipid lowering agents; antioxidants; extracellular matrix synthesis promoters; inhibitors of plaque inflammation and extracellular degradation; estradiol drug classes and their derivatives; proteins; vascular endothelial growth factor (VEGF) in any of its multiple isoforms; fibroblast growth factors; monocyte chemoatractant protein 1 (MCP-1); transforming growth factor alpha (TGF-alpha); transforming growth factor beta (TGF-beta) in any of its multiple isoforms; DEL-1, insulin like growth factors (IGF); placental growth factor (PLGF); hepatocyte growth factor (HGF); prostaglandin E1 (PG-E1); prostaglandin E2 (PG-E2); tumor necrosis factor alpha (TBF-alpha); granulocyte stimulating growth factor (G-CSF); granulocyte macrophage colony-stimulating growth factor (GM-CSF); angiogenin; follistatin; proliferin; genes encoding angiogenin, follistatin or proliferin; cells transfected with genes encoding angiogenin, follistatin or proliferin; pro-angiogenic peptides; PR39; PR11; pro-angiogenic small molecules and nicotine.
